(19) United States
(12) Patent Application Publication
(10) Pub. No.: US 2019/0029929 A1
(43) Pub. Date: Jan. 31, 2019

(54) PEROXID COMBINATIONS HAVING VISCOSITY

(71) Applicant: Naturegen Co., Ltd., Chungcheongbuk-do (KR)

(72) Inventor: Sug Youn Chang, Seoul (KR)

(21) Appl. No.: 16/071,515

(22) PCT Filed: Aug. 10, 2016
(86) PCT No.: PCT/KR2016/008772

§ 371 Date: Jul. 20, 2018

(30) Foreign Application Priority Data
Jan. 20, 2016 (KR) 10-2016-0007176
Aug. 9, 2016 (KR) 10-2016-0101011

(51) Int. Cl. A61K 8/02; A61K 8/25; A61K 8/22; A61K 8/81; A61K 8/34; A61Q 11/00

(52) U.S. Cl. CPC A61K 8/0208; A61K 8/25; A61K 8/22; A61K 2800/88; A61K 8/345; A61Q 11/00; A61K 2800/87; A61K 8/8176

(57) ABSTRACT

According to the present invention, a peroxide combination is prepared using silica particles. Therefore, the stability of a peroxide may be improved, and the amount of water used may be appropriately adjusted, so that the peroxide combination may be suitably used as a component of a whitening patch. In addition, since no separate drying process is required, cost competitiveness may be secured.

### TECHNICAL FIELD

The present invention relates to a peroxide combination and a patch-type tooth whitening product including the peroxide combination.

### BACKGROUND ART

As interest in tooth whitening has increased, various types of whitening toothpastes have become commercially available. However, even when a whitening agent having an excellent whitening effect is contained in toothpaste, it is difficult to confirm a significant whitening effect in a short period of time as a result of brushing teeth by using the toothpaste within a period of contact of 1 to 3 minutes. To solve this problem, in recent years, whitening products have been produced in various formulations, and patents related to whitening products have been filed.

For example, according to a method of P&G Co., a whitening product adherable to teeth, in which a gel-like peroxide is fixed on a polyethylene (PE) film having a well shape, can be produced at low cost (Patent Document 1). However, the product has weak adhesion to teeth at the time of use, and it is not easy to use the product because it sticks to the hands.

To overcome these problems, LG Household & Health Care Ltd. developed a dry film having a peroxide stabilized using a water-soluble glass polymer (Patent Document 2). The dry film has been used primarily in the manufacturing of tooth whitening products currently on the market.

However, when the aforementioned method is used, to dry a gel-like water-soluble polymer during the manufacturing process, the polymer must be passed through a long drying tunnel at a limited speed. Therefore, the production rate is limited, which may cause an increase in production costs. In addition, since a large amount of a water-soluble film former is used, the raw material cost may increase.

[Patent Document 1] U.S. Pat. No. 5,879,691

[Patent Document 2] Korean Patent Application Publication No. 10-2003-0005155

### DISCLOSURE

### Technical Problem

The present invention is directed to providing a peroxide combination prepared using silica particles. According to the present invention, the stability of a peroxide may be improved, and the amount of water used may be appropriately adjusted, so that the peroxide combination may be suitably used as a component of a tooth whitening patch. In addition, since no separate drying process is required, cost competitiveness may be obtained.

### Technical Solution

One aspect of the present invention provides a patch-type tooth whitening product including a peroxide combination including a liquid-phase peroxide, silica, and water, wherein the silica is fumed silica and is contained in an amount of 5 to 30 parts by weight based on 100 parts by weight of the peroxide combination, and the water is contained in an amount of 20 to 80 parts by weight based on 100 parts by weight of the peroxide combination.

### Advantageous Effects

The peroxide combination according to the present invention is prepared using silica particles. Therefore, the stability of the peroxide can be improved, and the amount of a liquid-phase peroxide diluent used can be appropriately adjusted, so that the peroxide combination can be suitably used as a component of a whitening patch. In addition, since no separate drying process is required, cost competitiveness can be secured.

### DESCRIPTION OF DRAWINGS

FIG. 1 is a photo of the peroxide combinations of Samples A\textcircled{1} to A\textcircled{3}.

FIG. 2 is a photo of the peroxide combinations of Samples 1 to 4.

FIG. 3 is a photo of patches prepared using the peroxide combinations of Samples K\textcircled{3}.1 and K\textcircled{3}.2.

FIG. 4 is a photo of a patch prepared using the peroxide combination of Sample 12.

### BEST MODE

The present invention relates to a patch-type tooth whitening product comprising a peroxide combination including a liquid-phase peroxide, silica, and water, wherein the silica is fumed silica and is contained in an amount of 5 to 30 parts by weight based on 100 parts by weight of the peroxide combination, and the water is contained in an amount of 20 to 80 parts by weight based on 100 parts by weight of the peroxide combination.

Hereinafter, the patch-type tooth whitening product of the present invention will be described in detail.

The patch-type tooth whitening product of the present invention includes a peroxide combination.

The peroxide combination may be a viscous material or a semi-solid phase material. Since the peroxide combination has fluidity, the peroxide combination may be spread on a substrate when applied to the patch, and may impart stickiness to the surface of the patch and the teeth.

The viscous material may have a viscosity of 50 to 200,000 cP, 5,000 to 200,000 cP, or 10,000 to 200,000 cP. The peroxide combination is formed within this range.

The semi-solid phase refers to a state in which a solid is formed without being completely dried up. When pressure is applied during the semi-solid phase, a film phase may be formed. When the peroxide combination is present in a semi-solid phase, the peroxide combination may have a viscosity greater than 200,000 cP. When the semi-solid phase combination is applied to a patch, the semi-solid phase combination may be spread on a substrate. The semi-solid phase combination may impart stickiness to the surface of the patch and the teeth. The upper limit of the viscosity is not particularly limited as long as the film phase may be formed upon application of pressure.

As described above, the peroxide combination according to the present invention includes a liquid-phase peroxide, silica, and water. In the combination, the liquid-phase peroxide may be adsorbed on the silica.

Since a solid peroxide component has a higher molecular weight than hydrogen peroxide, efficiency related to whitening is low, and a certain amount of time is required for elution. In addition, the solid peroxide component is less competitive than a liquid-phase peroxide in terms of commercially required factors such as manufacturing costs, convenience, and fast reaction time. Therefore, according to the present invention, a tooth whitening product may be produced using a liquid-phase peroxide.

In one embodiment, hydrogen peroxide or a peroxide solution containing one or more selected from the group consisting of carbamide peroxide, calcium peroxide, sodium percarbonate, sodium perborate, and tetrasodium pyrophosphate peroxidate may be used as the liquid-phase peroxide. In an embodiment of the present invention, hydrogen peroxide may be used as the liquid-phase peroxide. Since hydrogen peroxide has relatively low molecular weight, hydrogen peroxide has higher tooth whitening capacity than other substances having the same weight.

In one embodiment, the peroxide solution may be prepared by dissolving the peroxide in a powder form in a solvent such as water and ethanol.

In the present invention, the silica serves as a carrier for carrying the liquid-phase peroxide. The viscosity of silica is adjusted, and the silica is applied to a substrate to improve the maintaining of the shape maintenance of a patch.

When a liquid-phase peroxide, specifically hydrogen peroxide, is mixed with raw materials used for oral products, a reaction between the liquid-phase peroxide and the raw materials may occur. Thus, the stability of the liquid-phase peroxide may be reduced when used in the oral cavity. Therefore, in the present invention, the liquid-phase peroxide may be carried or adsorbed on silica, so that the stability of the liquid-phase peroxide may increase, and thus the liquid-phase peroxide may be easily applied to oral products such as a whitening patch.

In the present invention, fumed silica may be used as the silica. The fumed silica has a high absorption capability and is suitable as a carrier used in capsules.

In the present invention, the content of the liquid-phase peroxide is not particularly limited, and may be changed based on the level of approval of each country when the liquid-phase peroxide is commercialized. For example, the content of the liquid-phase peroxide may be 0.01 to 25 parts by weight, 0.1 to 20 parts by weight, or 1 to 20 parts by weight based on 100 parts by weight of the peroxide combination. The above-described content represents a case wherein peroxide is converted into hydrogen peroxide. Within this range, a whitening effect may be excellent, and stability may be improved. The concentration of the liquid-phase peroxide may be adjusted using purified water to obtain the desired content.

The content of the silica is not particularly limited, and may be 5 to 30 parts by weight, 7 to 25 parts by weight, 10 to 20 parts by weight, or 10 to 18 parts by weight based on 100 parts by weight of the peroxide combination. Within this range, the peroxide combination may be formed.

In addition, water contained in the peroxide combination of the present invention may be purified water. The content of the water may be 20 to 80 parts by weight, 30 to 80 parts by weight, or 40 to 80 parts by weight based on 100 parts by weight of the peroxide combination. The peroxide combination may contain an excess amount of water to improve ease of adhesion and desorption.

In the present invention, in addition to the liquid-phase peroxide, silica, and water described above, the peroxide combination may additionally include water-soluble polymers. The water-soluble polymers may aggregate silica, and may also impart chemical and physical stability to a peroxide combination that is being prepared.

The type of water-soluble polymer is not particularly limited, and the water-soluble polymer may include, for example, one or more selected from the group consisting of polyvinylpyrrolidone (PVP), polyvinyl alcohol (PVA), carboxymethyl cellulose (CMC), polyethylene glycol (PEG), quaternary ammonium polymers, carboxypolymethylene, carboxypropyl cellulose, poloxamers, carrageenan, carboxyvinyl polymers, karaya, xanthan gum, guar gum, arabic gum, polyvinylpyrrolidone/vinyl acetate copolymers, hydroxypropyl cellulose, hydroxypropylmethyl cellulose, hydroxyethyl cellulose, hydroxypropylethyl cellulose, Polyox, Carbopol, Polyquaternium-11, Polyquaternium-39, and polyalkyl vinyl ether/maleic acid copolymers (PVM/MA copolymers: Gantrez AN 119; AN 139; and S-97). Specifically, polyvinylpyrrolidone (PVP), polyvinyl alcohol (PVA), carboxymethyl cellulose (CMC), polyethylene glycol (PEG), and quaternary ammonium polymers may be used as the water-soluble polymer.

In the present invention, the content of the water-soluble polymer is not particularly limited, and may be changed depending on the content of silica and the type of polymer. For example, the content of the water-soluble polymer may be 3 to 70 parts by weight, 5 to 60 parts by weight, 5 to 50 parts by weight, 5 to 40 parts by weight, or 5 to 30 parts by weight based on 100 parts by weight of the peroxide combination. Within this range, the polymer may be easily mixed with other components, and a peroxide combination having excellent stability and stickiness may be prepared.

In addition, in the present invention, the peroxide combination may additionally include a moisturizer. The moisturizer is a component capable of retaining moisture and may prevent the combination from drying during distribution. In addition, the moisturizer may serve as a humectant or plasticizer.

The type of moisturizer is not particularly limited, and the moisturizer may be, for example, one or more selected from the group consisting of glycerin, ethylene glycol, and polymers thereof as well as hyaluronic acid and an aloe extract, which have moisture retention capability.

The content of the moisturizer may vary depending on the content of silica. For example, the content of the moisturizer may be 0.1 to 20 parts by weight or 3 to 15 parts by weight based on 100 parts by weight of the peroxide combination. Within this range, drying of the combination during distribution may be prevented. When the content is less than 0.1 part by weight, the surface of the patch may become dry, and the combination may break up when the patch is stored for a long period of time. When the content is greater than 20 parts by weight, the surface of the patch becomes excessively damp, and it is difficult to maintain the shape of the patch, which may cause inconvenience in use.

In the present invention, the peroxide combination may additionally include a chelating agent selected from the group consisting of tetrasodium pyrophosphate (TSPP) and sodium acid pyrophosphate (SAPP), as pyrophosphates; and sodium hexametaphosphate (SHMP), sodium tripolyphosphate (STPP), an acidic polyphosphate (APP, e.g., Sporix™ (Shodo Chemical Co.)), and mixtures thereof, as polyphosphates.

When the chelating agent TSPP, SAPP, SHMP, or STPP is used with a peroxide, a synergistic effect may be obtained in terms of whitening effect compared to a case wherein a peroxide and the chelating agent are used separately.

In addition, in the present invention, the peroxide combination may additionally include enzymes (specifically, one or more of dextranase and glucose oxidase) that involve difficulty in terms of being applied to toothpaste due to stability concerns. In addition, the peroxide combination may additionally include triclosan, chlorohexidine, or vitamin E or a derivative thereof as a medicinal ingredient for treatment of oral diseases, or a peroxide or chlorophyll or a derivative thereof to remove bad breath.

The peroxide combination according to the present invention may be prepared using various methods known in the art.

In one embodiment, the combination may be prepared through a preparation step for the combination, in which a polymer is dissolved in the liquid-phase peroxide using a mixer, and then uniformly mixed with fumed silica.

The preparation step for the combination is responsible for preparing a combination having tooth whitening efficacy. In the preparation step for the combination, the above-described water-soluble polymer may be used as the polymer, and the above-described peroxide may be used as the liquid-phase peroxide.

In addition, the present invention relates to a patch-type tooth whitening product including the above-described peroxide combination.

In one embodiment, the patch-type tooth whitening product may be a finished product including a substrate to which the peroxide combination is applied.

In addition, in one embodiment, the patch-type tooth whitening product may consist of the substrate and the peroxide. In this case, the substrate and the peroxide combination may be provided separately from each other. The peroxide combination may be applied to the substrate at the time of use. In addition, a container, such as a tube, filled with the peroxide combination may be provided.

In the present invention, the substrate may serve as a support layer used in a typical patch-type tooth whitening product. The substrate to which the peroxide combination is applied may serve to provide a support layer for the peroxide combination, and may improve stability.

In the present invention, the substrate may be a film or a non-woven fabric. For example, polyethylene, polypropylene, and polyethylene terephthalate (PET) may be used as the film. As the non-woven fabric, a non-woven fabric made of a material used in the film or a hydrophilic non-woven fabric may be used.

The hydrophilic non-woven fabric may be made of rayon, cotton, pulp, pulp/melt-blown, pulp/spunbond, or mixtures thereof.

A hydrophobic film or non-woven fabric may be bonded to one surface of the hydrophilic non-woven fabric to which the peroxide combination is not applied. When the hydrophilic non-woven fabric is used, the peroxide combination attached to a substrate may be diluted due to infiltrated saliva. As a result, the effect of the peroxide combination may be reduced. Therefore, the shape of the patch may be protected by bonding the hydrophobic film or non-woven fabric. In this case, the hydrophobic non-woven fabric or film may be made of polypropylene, polyethylene terephthalate, polypropylene/polyethylene/polyester, or mixtures thereof. In addition, the hydrophobic non-woven fabric or film may be made of one or more low water-soluble polymers selected from the group consisting of ethyl cellulose, Eudragit, copolymers of poly(dl-lactic acid) or poly(dl-lactic/glycolic acid), poly-ε-caprolactone, hydroxypropyl methylcellulose or derivatives thereof, polymers of polymethyl acrylic acid and acrylic acid, cellulose acetate or derivatives thereof, cellulose phthalate trimellitate, ethyl cellulose, and methyl cellulose.

In addition, for example, the surface of a substrate to which the peroxide combination is applied may be modified with the hydrophilic polymer. In this case, the hydrophilic polymers exemplified above may be used without limitation.

In addition, in the present invention, elastic materials such as silicone rubber, polyurethane rubber, and polybutadiene rubber may be used as the substrate. The substrate may have pores.

The thickness of the substrate is not particularly limited, and may be 10 μm to 1 mm, 10 to 500 μm, 10 to 100 μm, or 20 to 50 μm. Within this range, the substrate may be easily prepared, the peroxide combination may be easily applied to the substrate, and the discomfort that may arise in the oral cavity may be reduced.

In the present invention, the substrate may additionally include a plasticizer. castor oil, hydrogenated castor oil, polyethylene glycol or glycerin, and the like, which may be used as the plasticizer.

In addition, to achieve visual whitening as well as whitening by chemical and physical activity when the substrate is attached to the teeth, a white pigment, such as titanium dioxide, talc, hydroxyapatite, and zinc oxide, may be used in combination with the substrate, or the substrate may be coated with the white pigment to form a separate layer. When the pigments are not compatible with peroxides, surface-treated titanium dioxide may be used. In addition to white pigments, pearl pigments and pigments of various colors may be applied as desired.

In the present invention, the thickness of the peroxide combination applied to the substrate may be appropriately adjusted based on the use and other factors.

### MODES OF THE INVENTION

The advantages and features of the present invention and the manner of achieving them will become apparent with reference to the embodiments described in detail below. The invention may, however, be embodied in many different forms and should not be construed as being limited to the embodiments set forth herein; rather, these embodiments are provided so that this disclosure will be thorough and complete, and will fully convey the concept of the invention to one of ordinary skill in the art. The invention is defined by the scope of the claims of the invention.

### EXAMPLES

### Preparation Example 1

### Evaluation of Properties of Peroxide Combination According to Content of Fumed Silica

Peroxide combinations (A\textcircled{1} to A\textcircled{3}) were prepared according to the compositions and contents shown in Table 1 below.

Specifically, 35% hydrogen peroxide, polyvinylpyrrolidone (PVP) K90, purified water, and glycerin were added to an Erlenmeyer flask and stirred using a stirrer until PVP was completely dissolved in order to prepare a mixed solution.

Thereafter, the mixed solution was added to fumed silica (Aerosil, Evonik Co.) and kneaded until the solution and fumed silica were evenly mixed in order to prepare a combination.

TABLE 1

| | Sample A [symbol] | Sample A [symbol] | Sample A [symbol] |
|---|---|---|---|
| Purified water | 74 | 72 | 70 |
| H₂O₂ (35%) | 9 | 9 | 9 |
| PVP K90 | 8 | 8 | 8 |
| Glycerin | 6 | 6 | 6 |
| Aerosil | 3 | 5 | 7 |

The viscosity and physical properties of prepared Samples A\textcircled{1} to A\textcircled{3} were evaluated.

The viscosity was measured by the following method using a Brookfield viscometer.

1. Using a spindle no. 7 and allowing the spindle to lightly touch the surface of the sample before starting measurement.

2. Placing the spindle into the sample to a depth of about 50 mm (at this time, a fixed viscosity measurement value is roughly observed).

3. Reading a viscosity value when the spindle reaches the correct measurement position.

In addition, the physical properties were visually evaluated.

The measurement results are shown in Table 2 below.

TABLE 2

| | Sample A [symbol] | Sample A [symbol] | Sample A [symbol] |
|---|---|---|---|
| Viscosity | 4,000 cP | 22,000 cP | 102,000 cP |
| Physical properties | Diluted | Combination is | Combination is |
| | | formed | stable |

As shown in Table 2, it can be seen that Samples A\textcircled{2} to A\textcircled{3} prepared according to Preparation Example 1 are formed as a combination.

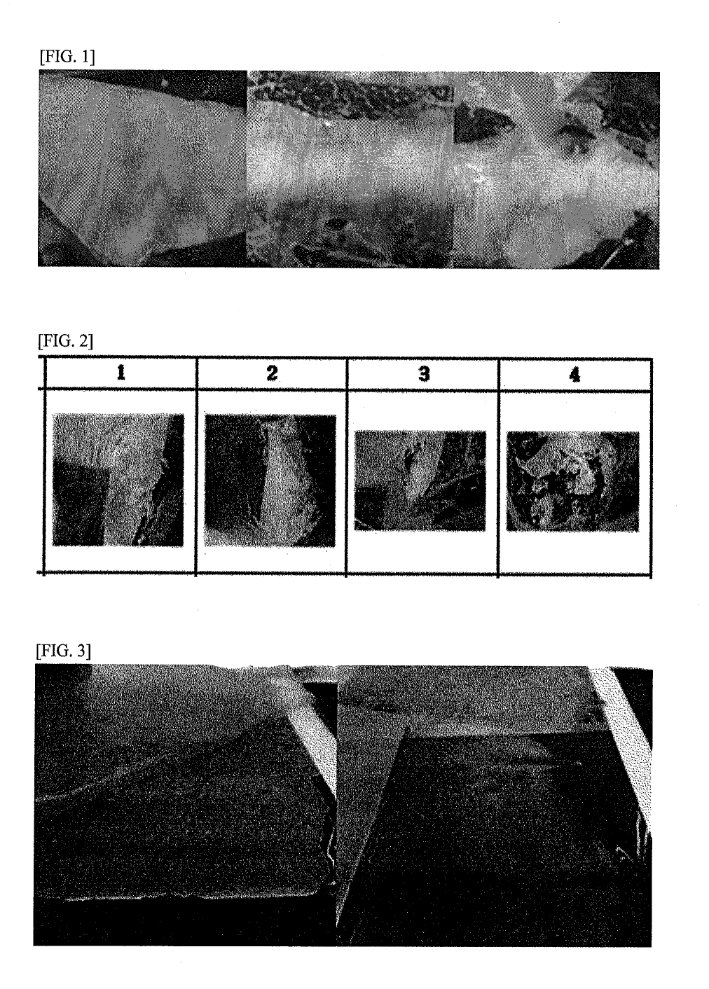

In addition, FIG. 1 is photo of the combination of Samples A\textcircled{1} to A\textcircled{3}. As shown in FIG. 1, it can be seen that combinations are formed (A\textcircled{1}; left image, A\textcircled{2}; middle image, and A\textcircled{3}; right image).

In the case of Sample A\textcircled{1}, as shown in the photo of FIG. 1 and the physical property evaluation results, the combination was diluted, and thus the combinations could not be applied to a patch. That is, it can be seen that the combinations may be easily prepared when the amount of silica used exceeds 5 parts by weight.

### Preparation Example 2

### Evaluation of Ease of Mixing According to Content of Water-Soluble Polymer

Combinations 1 to 4 were prepared according to the compositions and contents shown in Table 3 below.

The combinations were prepared in the same manner as in Preparation Example A\textcircled{1}, except that a peroxide was not used.

TABLE 3

| | Sample 1 | Sample 2 | Sample 3 | Sample 4 |
|---|---|---|---|---|
| Purified water | 10 | 20 | 30 | 40 |
| PVP K90 | 75 | 65 | 55 | 45 |
| Glycerin | 10 | 10 | 10 | 10 |
| Aerosil | 5 | 5 | 5 | 5 |

As a result of preparation of the combinations, based on the case wherein the content of fumed silica (Aerosil) was 5 parts by weight, when the content of a water-soluble polymer (PVP K90) was 75 parts by weight, mixing was made very difficult; when the content of the water-soluble polymer (PVP K90) was 65 parts by weight, effort and time were required to perform mixing; when the content of the water-soluble polymer (PVP K90) was 55 parts by weight, mixing was somewhat difficult; and when the content of the water-soluble polymer (PVP K90) was 45 parts by weight, mixing was made easy.

In the present invention, referring to the photo in FIG. 2, it can be seen that the composition of Sample 4 is easily mixed.

### Preparation Example 3

### Preparation of Peroxide Combination

Peroxide combinations (J\textcircled{1} to J) were prepared according to the compositions and contents (parts by weight) of Table 4 below.

The peroxide combinations were prepared in the same manner as in Preparation Example A\textcircled{1}, and the prepared samples (peroxide combinations) were applied to a PE film.

TABLE 4

| | Sample | Sample | Sample | | Sample | Sample | Sample | Sample |
|---|---|---|---|---|---|---|---|---|
| | J [symbol] | J [symbol] | J [symbol] | Sample J | K [symbol] .1 | K [symbol] .2 | K [symbol] .1 | K [symbol] .2 |
| Purified | 67 | 67 | 68 | 67 | 63 | 65 | 63 | 64 |
| water | | | | | | | | |
| H₂O₂ (35%) | 8 | 8 | 8 | 8 | 8 | 8 | 8 | 8 |
| PVP K90 | 7 | 7 | 7 | 7 | 7 | 8 | 8 | 8 |
| Glycerin | 5 | 5 | 5 | 5 | 9 | 9 | 8 | 9 |
| Aerosil | 13 | 12 | 11 | 13 | 13 | 11 | 13 | 11 |

The stability, viscosity, and physical properties of the prepared samples were evaluated.

Stability was measured after storage at 60° C. for 5 days.

Viscosity was measured using the above-described method.

In addition, whether each sample was uniformly applied to a substrate was evaluated, and the evaluation result was expressed in terms of uniform spreadability.

The degree of adhesion was evaluated when the sample was applied to the teeth, and the evaluation result was expressed in terms of feeling of adhesiveness during use.

When the uniform spreadability and feeling of adhesiveness during use were tested in 10 subjects, the results were expressed as 1: very bad, 2: bad, 3: normal, 4: good, and 5: very good.

The measured results are shown in Table 5 below.

TABLE 5

| | Sample | Sample | Sample | | Sample | Sample | Sample | Sample |
|---|---|---|---|---|---|---|---|---|
| | J [symbol] | J [symbol] | J [symbol] | Sample J | K [symbol] .1 | K [symbol] .2 | K [symbol] .1 | K [symbol] .2 |
| Stability | 98% | 96% | 97% | 95% | 96% | 97% | 98% | 97% |
| Viscosity | >200,000 | 197,000 | 182,000 | 196,000 | >200,000 | >200,000 | >200,000 | >200,000 |
| Uniform | 3 | 3 | 4 | 5 | 5 | 5 | 5 | 5 |
| spreadability* | | | | | | | | |
| Feeling | 4 | 4 | 5 | 3 | 5 | 4 | 5 | 4 |
| of | | | | | | | | |
| adhesiveness | | | | | | | | |
| during | | | | | | | | |
| use* | | | | | | | | |

As shown in Table 5, it can be seen that the peroxide combinations prepared according to the present invention have excellent uniform spreadability, feeling of adhesiveness during use, and stability.

In addition, in the present invention, FIG. 3 is photo of patches prepared using the peroxide combinations corresponding to Sample K\textcircled{3}.1 (left image) and Sample K\textcircled{3}.2 (right image), respectively.

As shown in FIG. 3, it can be seen that the peroxide combination according to the present invention may be easily applied to a patch.

### Preparation Example 4

### Preparation of Peroxide Combination

Peroxide combinations 8, 10, and 12 were prepared according to the compositions and contents (parts by weight) shown in Table 6 below.

The peroxide combinations were prepared in the same manner as in Preparation Example A\textcircled{1}. The prepared combination was applied to a polyethylene (PE) film having a thickness of 20 to 50 μm, so that the PE was coated with the peroxide combination.

Thereafter, the peroxide-coated PE film was attached to a release agent-coated polyethylene terephthalate (PET) film.

TABLE 6

| | Sample 12 |
|---|---|
| Purified water | 56.2 |
| H₂O₂(35%) | 8.0 |
| PVP K90 | 13 |
| Glycerin | 8.0 |
| Aerosil | 14.8 |

The releasability of the film of the prepared samples was evaluated.

The releasability of the film was evaluated by visually discriminating whether the peroxide combination coated on the PE film stably remains when the user removes the PET film.

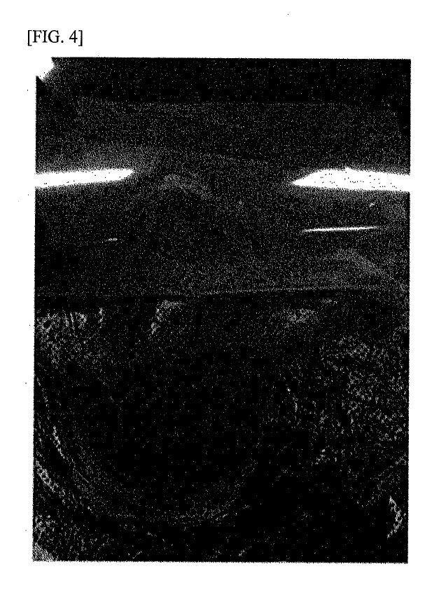

In the present invention, referring to images of the prepared Samples in FIG. 4, it can be seen that Sample 12 has excellent releasability of the film.

### INDUSTRIAL APPLICABILITY

The peroxide combination according to the present invention is prepared using silica particles. Therefore, the stability of a peroxide can be improved, and the amount of a liquid-phase peroxide diluent used can be appropriately adjusted, so that the peroxide combination can be suitably used as a component of a whitening patch. In addition, since no separate drying process is required, cost competitiveness can be secured.

1. A patch-type tooth whitening product, comprising a peroxide combination comprising a liquid-phase peroxide, silica, and water,
wherein the silica is fumed silica and is contained in an amount of 5 to 30 parts by weight based on 100 parts by weight of the peroxide combination; and
the water is contained in an amount of 20 to 80 parts by weight based on 100 parts by weight of the peroxide combination.

2. The patch-type tooth whitening product according to claim 1, wherein the peroxide combination is a viscous material having a viscosity of 50 to 200,000 cP or a semi-solid phase material that has a viscosity of greater than 200,000 cP and forms a film phase upon application of pressure.

3. The patch-type tooth whitening product according to claim 1, wherein the liquid-phase peroxide is hydrogen peroxide, or a solution containing one or more selected from the group consisting of carbamide peroxide, calcium peroxide, sodium percarbonate, sodium perborate, and tetrasodium pyrophosphate peroxidate.

4. The patch-type tooth whitening product according to claim 1, wherein the peroxide combination further comprises water-soluble polymers.

5. The patch-type tooth whitening product according to claim 4, wherein the water-soluble polymers comprises one or more selected from the group consisting of polyvinylpyrrolidone (PVP), polyvinyl alcohol (PVA), carboxymethyl cellulose (CMC), polyethylene glycol (PEG), quaternary ammonium polymers, carboxypolymethylene, carboxypropyl cellulose, poloxamers, carrageenan, carboxyvinyl polymers, karaya, xanthan gum, guar gum, arabic gum, polyvinylpyrrolidone/vinyl acetate copolymers, hydroxypropyl cellulose, hydroxypropylmethyl cellulose, hydroxyethyl cellulose, hydroxypropylethyl cellulose, Polyox, Carbopol, Polyquaternium-11, Polyquaternium-39, and polyalkyl vinyl ether/maleic acid copolymers (PVM/MA copolymers: Gantrez AN 119; AN 139; and S-97).

6. The patch-type tooth whitening product according to claim 1, wherein the peroxide combination further comprises one or more moisturizers selected from the group consisting of glycerin, ethylene glycol, and polymers thereof and hyaluronic acid and an aloe extract.

7. The patch-type tooth whitening product according to claim 6, wherein the moisturizers are contained in an amount of 0.1 to 20 parts by weight based on 100 parts by weight of the peroxide combination.

8. The patch-type tooth whitening product according to claim 1, wherein the patch-type tooth whitening product consists of a substrate and the peroxide combination applied to the substrate.

9. The patch-type tooth whitening product according to claim 1, wherein the patch-type tooth whitening product consists of a substrate and the peroxide combination, wherein the substrate and the peroxide combination are provided separately from each other, and the peroxide combination is applied to the substrate when the patch-type tooth whitening product is used.

10. The patch-type tooth whitening product according to claim 8 or 9, wherein the substrate is a non-woven fabric or a film.
